(19) United States
(12) Patent Application Publication
(10) Pub. No.: US 2024/0148301 A1
(43) Pub. Date: May 9, 2024

(54) SMART WEARABLE DEVICE

(71) Applicant: aiFree Interactive Technology CO., LTD., Tainan City (TW)

(72) Inventors: Chien-Hsiang Chang, Tainan City (TW); Yang-Cheng Lin, Tainan City (TW); Wei-Chih Lien, Tainan City (TW); Tseng-Ping Chiu, Tainan City (TW); Pei-Yun Wu, Tainan City (TW); Bo Liu, Tainan City (TW)

(21) Appl. No.: 17/981,897

(22) Filed: Nov. 7, 2022

(51) Int. Cl. A61B 5/256; A61B 5/296

(52) U.S. Cl. CPC A61B 5/256; A61B 5/296; A61B 2560/0228

(57) ABSTRACT

The present invention provides a smart wearable device, which is held on an upper body of a wearer by a plurality of contact pad sets, and has a connection unit, a first sensing module, a second sensing module, and an extension unit.

### BACKGROUND OF THE INVENTION

### 1. Field of the Invention

The present invention generally relates to a wearable device, more particularly to a smart wearable device, which is held on an upper limb/body of a wearer by a plurality of contact pad sets.

### 2. Description of the Prior Art

Population aging has become an important social issue in the world. Recently, the problem of “sarcopenia” of the elders is being paid much attention. As the ages go by, the size and strength of muscles gradually decline, leading to a downward trend in overall physiological functions and resulting in weakened muscles symptoms, thereby increasing the risk of death and extending multiple complications. Since the core concept of sarcopenia is the loss of skeletal muscle, which is more obvious than the normal aging process. For further worse conditions' considerations, it affects the performance of muscle function, so as to result in limitation of mobility, and often faces inconveniences in performing activities of daily living, further causing negative health effects.

Presently, elderly people or patients with sarcopenia are allowed to perform resistance exercise training to increase muscle mass or muscle strength. However, general resistance exercise trainings may usually go to a gym or a place with gravity training equipment. It is not easy for the elders with functional degrading problems, and the number of training sessions is easily limited by the manpower or space of professional trainers as well. In addition, with the appearance of an advance age society, rehabilitation patients and sub-health groups are increasing gradually. Debilitating disease is very common among the elders, and it raises the risk of falls and other diseases also taking place. Generally, rehabilitation training is able to help the recovery of limb motor function. The traditional one-on-one training may need a large amount of therapists. The current gap in the country may not be figured out in a short term. On the other hand, the rehabilitation process is boring and short of the initiative of the brain motor nerves that control the limbs. Therefore, such participation leads to ineffective rehabilitation.

In the age of 21th century and information explosion era, the speedy transition of science is rapidly changing the living model of human beings, thereby the development of new technologies such as smart medical care and smart sensing and the popularization of customized health care concepts are spread out rapidly, and aforesaid items become one of the fields of medical development prospects. The medical development and application in the aspect of wearable device, including dataization of behavioral functions and health monitoring contents, are being achieved the effect of medical prevention and health care. Moreover, they are even applied to the field of medical rehabilitation, in order to promote effect and efficiency for the convenience, safety and comfort of user.

The integration technology for the wearable device and information communication provides patients with sarcopenia, frailty, and elderly groups a remote and digital service system for supplying the most optimal rehabilitation plans, in order to reduce the user's experience of fatigue, and improve long-term rehabilitation training motivation for resonating the elders and reducing the negative impact of aging.

### SUMMARY OF THE INVENTION

The main purpose of the present invention provides a smart wearable device, which is held on an upper body of a wearer by a plurality of contact pad sets, and has a connection unit, a first sensing module, a second sensing module, and an extension unit.

The connection unit, having a connection band that detachably holds on the upper body of the wearer, a first positioning slot disposed on the connection band, a first positioning set disposed on the first positioning slot, a second positioning slot disposed on the connection band, and a second positioning set disposed on the second positioning slot. The connection band has a first end and a second end opposite to the first end. A horizontal line is formed from the first end to the second end. A vertical line is vertical to the horizontal line. The first positioning slot is at the first end. The second positioning slot is at the second end.

Preferably, the first positioning set has a first positioning hole, a second positioning hole, and a third positioning hole. A first set point is defined based on the first positioning slot as a center. The first positioning hole, the second positioning hole, and the third positioning hole are disposed around the first set point. The first positioning hole is on the horizontal line. The second positioning hole and the third positioning hole are disposed up and down along the vertical line, and located on one side of the first positioning hole.

Preferably, the second positioning set has a fourth positioning hole, a fifth positioning hole, and a sixth positioning hole. A second set point is defined based on the second positioning slot as a center. The fourth positioning hole, the fifth positioning hole, and the sixth positioning hole are disposed around the second set point.

Preferably, the fourth positioning hole is on the horizontal line, and the fifth positioning hole and the sixth positioning hole are disposed up and down along the vertical line, and located on one side of the fourth positioning hole.

Preferably, the first positioning slot and the second positioning slot are disposed on the horizontal line at a bilateral symmetrical interval, and the first positioning set and the second positioning set are disposed at a bilateral symmetrical interval on the horizontal line.

Preferably, the first positioning slot has a first flange that protrudes outwardly along a peripheral of the first positioning slot and is based on the first set point as a center. The first flange is disposed close to the second positioning hole and the third positioning hole. The second positioning slot has a second flange that protrudes outwardly along a peripheral of the second positioning slot and is based on the second set point as a center. The second flange is disposed close to the fifth positioning hole and the sixth positioning hole.

Preferably, the connection band further has a plurality of alignment calibration lines at the second end. The plurality of alignment calibration lines are spaced apart from each other and arranged along the vertical line.

Preferably, the connection band further has a first joint member at the first end and a second joint member at the second end. The first joint member and the second joint member are connected with each other in order to hold the connection band on the upper body of the wearer.

Preferably, the first sensing module further has a first physiological sensing circuit, and the second sensing module further has a second physiological sensing circuit. The first physiological sensing circuit, the second physiological sensing circuit and a multimedia interactive system are connected with each other in wireless.

Preferably, the first sensing set has a first sensing point connecting with the first positioning hole, a second sensing point connecting with the second positioning hole and a third sensing point connecting with the third positioning hole. The second sensing set has a fourth sensing point connecting with the fourth positioning hole, a fifth sensing point connecting with the fifth positioning hole, and a sixth sensing point connecting with the sixth positioning hole.

Preferably, a third set point is defined based on the first extension block as a center, and a fourth set point is defined based on the second extension block as a center. The first fastening set has a first fastening member, a second fastening member, and a third fastening member. The first fastening member, the second fastening member and the third fastening member are disposed around the third set point. The second fastening set has a fourth fastening member, a fifth fastening member and a sixth fastening member. The fourth fastening member, the fifth fastening member and the sixth fastening member are disposed around the fourth set point. The fourth fastening member is connected with the first sensing point. The fifth fastening member is connected with the second sensing point. The sixth fastening member is connected with the third sensing point.

Preferably, the extension band is on the vertical line, and the first fastening member, the second fastening member and the third fastening member are rotated 30 to 50 degrees clockwise with respect to the fourth fastening member, the fifth fastening member, and the sixth fastening member based on the third setting point as an axis when the connection band is connected with the extension band.

Preferably, the fourth positioning hole, the fifth positioning hole and the sixth positioning hole are rotated 15 to 30 degrees clockwise with respect to the first positioning hole, the second positioning hole and the third positioning hole based on the second setting point as an axis.

The advantages of the present invention are described as following. To capture the muscle activities of the deltoid is through the first extension block and the second sensing module, and the muscle activities of the biceps are obtained by the first sensing module, in order to detect the muscle activities of several parts in the upper limb/body. It also stores the activity of each muscle group of the user during exercise training, and conforms to the human factor design and the human-oriented scale record design as the main axis. The plurality of alignment calibration lines are spaced apart from each other on the upper limb/body of the wearer for universal design trends. The first flange and the second flange are configured to be two fool proof settings for guiding the wearer to place the first sensing module and the second sensing module. Further, the physiological sensing data and the muscle activities captured by the first physiological sensing circuit and the second physiological sensing circuit are delivered outwardly for analysis to integrate the wearable device with the information, so as to provide sarcopenia, frailty, and elderly groups with remote and digital service systems, accurately afford the most optimal rehabilitation plan, reduce the user's experience of fatigue, and improve long-term rehabilitation training motivation.

Other and further features, advantages, and benefits of the invention will become apparent in the following description taken in conjunction with the following drawings. It is to be understood that the foregoing general description and following detailed description are exemplary and explanatory but are not to be restrictive of the invention. The accompanying drawings are incorporated in and constitute a part of this application and, together with the description, serve to explain the principles of the invention in general terms. Like numerals refer to like parts throughout the disclosure.

### BRIEF DESCRIPTION OF THE DRAWINGS

The objects, spirits, and advantages of the preferred embodiments of the present invention will be readily understood by the accompanying drawings and detailed descriptions, wherein:

FIG. 1 illustrates a schematic view of a first preferred embodiment of the smart wearable device of the present invention;

FIG. 2 illustrates a schematic view of the first preferred embodiment held on a wearer's upper limb;

FIG. 3 illustrates a schematic view of a first sensing module and a second sensing module located on a connection unit of the first preferred embodiment;

FIG. 4 illustrates a schematic view of a first end and a second end in the connection unit connected with each other of the first preferred embodiment;

FIG. 5 illustrates a schematic view of another view angle of the first preferred embodiment;

FIG. 6 illustrates a schematic view of an extension unit of the first preferred embodiment;

FIG. 7 illustrates a schematic view of the first preferred embodiment without the first sensing module and the second sensing module;

FIG. 8 illustrates a schematic view of a second preferred embodiment of the smart wearable device of the present invention; and

FIG. 9 illustrates a schematic view of an extension unit of the second preferred embodiment.

### DETAILED DESCRIPTION OF THE INVENTION

Following preferred embodiments and figures will be described in detail so as to achieve aforesaid objects.

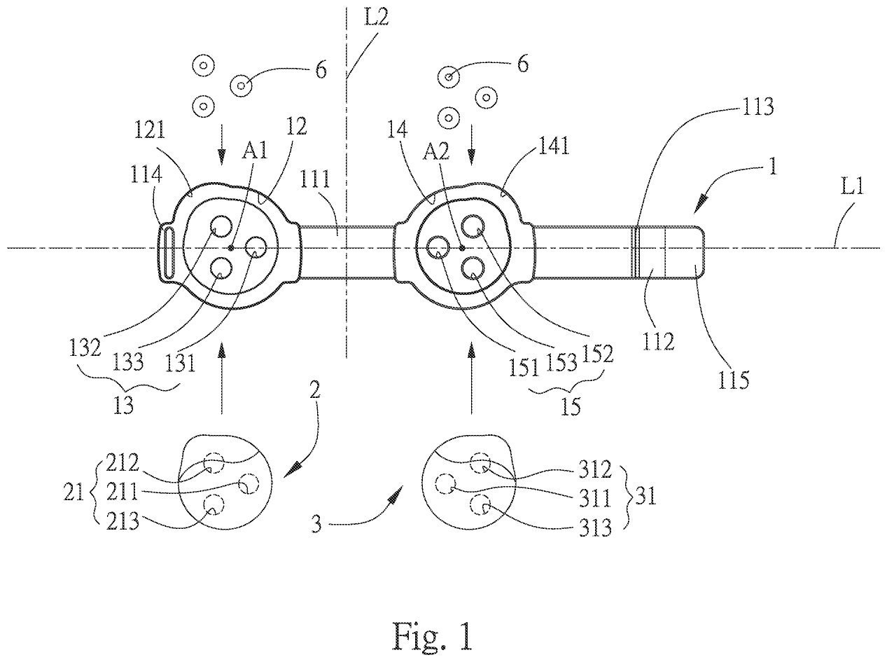

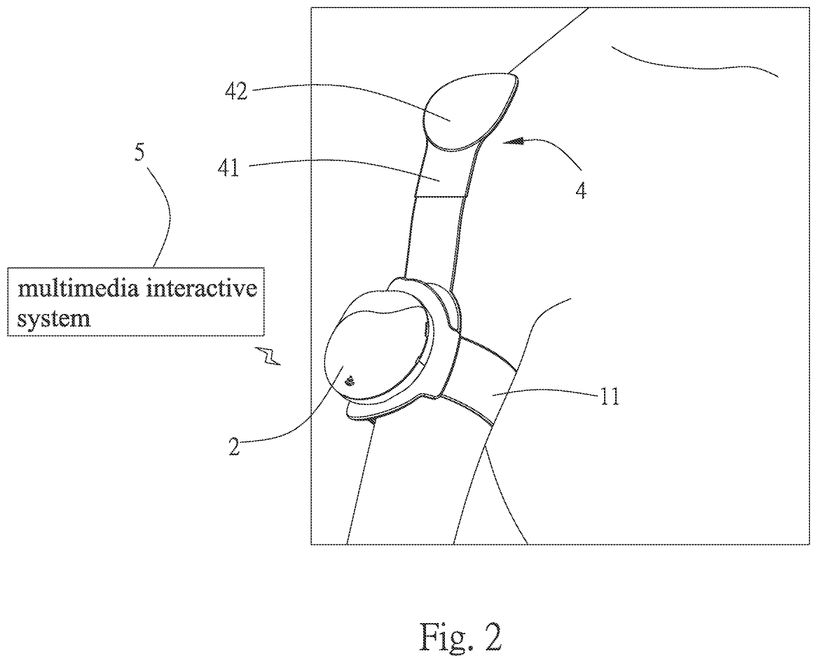

With reference to FIG. 1 and FIG. 2, which illustrate a schematic view of a first preferred embodiment of the wearable device of the present invention. The wearable device is held on an upper body of a wearer by a plurality of contact pad sets 6, and includes a connection unit 1, a first sensing module 2, a second sensing module 3, and an extension unit 4. Each contact pad set 6 has three contact pads for remaining the wearable device on the upper body of the wearer. The wearable device is designed to combine comfortable and easy-to-wear products, and can effectively record the values, directions and speeds of multiple muscle group movements.

The connection unit 1 includes a connection band 11 that detachably holds on the upper body of the wearer, a first positioning slot 12 disposed on the connection band 11, a first positioning set 13 disposed on the first positioning slot 12, a second positioning slot 14 disposed on the connection band 11, and a second positioning set 15 disposed on the second positioning slot 14.

The connection band 11 includes a first end 111 and a second end 112 opposite to the first end 111. A horizontal line L1 is formed from the first end 111 to the second end 112. A vertical line L2 is vertical to the horizontal line L1. The first positioning slot 12 is at the first end 111, and the second positioning slot 14 is at the second end 112. The horizontal line L1 is defined as a length direction of the connection band 11, and the vertical line L2 is defined as a width direction of the connection band 11, wherein the horizontal line L1 is not at a middle position of the width direction of the connection band 11.

In practice, the contact pad may be applied by different usages. For example, in the form of active therapy devices, such as neuromuscular electrical stimulation (NMES) pads, thermal therapy pads, in the form of pain relief patches in transdermal therapy pads, or in the form of acupuncture patches. In applications of capturing range-of-motion/motion-tracking data, the contact pads can take the form of precisely placed physiological sensor components configured as corresponding external add-on components to extract or otherwise obtain physiological information associated with the wearer's body part information (such as tracking of muscle groups, range of motion, range of motion, load, etc.).

The contact pad is an electromyographic patch for measuring electromyographic signals. The contact pad attached to the skin on the surface of the muscle is to measure the voltage variations of the muscle during activity. When the electromyographic signal is larger, the more motor units participate in the muscle activity, and the higher the degree of stimulation, more, the degree of local muscle activity is inferred by electromyography (EMG). Through capturing surface electromyographic signals and six-axis inertial sensing technology, the musical groups of the active conditions of the wearer during activity training are effectively detected and stored. The obtained related information will be stored in a host device, and then processed by backend systems for data analysis and evaluation.

The connection band 11 further has a plurality of alignment calibration lines 113 at the second end 112, and the plurality of alignment calibration lines 113 are spaced apart from each other and arranged along the vertical line L2. In addition, the connection band 11 further has a first joint member 114 at the first end 111 and a second joint member 115 at the second end 112. The first joint member 114 and the second joint member 115 are connected with each other in order to hold the connection band 11 on the upper body of the wearer.

The plurality of alignment calibration lines 113 are space apart from each other to dispose on the upper body of the wearer for interval adjustment, so as to fit different users and approach universal design trends. More, the first joint member 114 and the second joint member 115 are connected with each other to form a ring type, therefore the connection unit 1 takes the attitude of self-preservation to be around the upper body of the wearer. The first joint member 114 has a through slot, and the second joint member 115 has a Velcro/hook and loop tape. Thus, the second joint member 115 goes through the through slot, so the Velcro/hook and loop tape is stick on the connection band 11. In practical implementation, the first and second joint members 114 and 115 can be configured as a button, a magnetic attraction, an adhesive, a tenon, or an elastic band structure to engage with each other on the wearer's upper limbs.

The first positioning slot 12 and the second positioning slot 14 are disposed on the horizontal line L1 at a bilateral symmetrical interval, and the first positioning set 13 and the second positioning set 15 are disposed at a bilateral symmetrical interval on the horizontal line L1.

The first positioning set 13 has a first positioning hole 131, a second positioning hole 132, and a third positioning hole 133. A first set point A1 is defined based on the first positioning slot 12 as a center. The first positioning hole 131, the second positioning hole 132 and the third positioning hole 133 are disposed around the first set point A1. The first positioning hole 131 is on the horizontal line L1, and the second positioning hole 132 and the third positioning hole 133 are disposed up and down along the vertical line L2, and located on one side of the first positioning hole 131.

The second positioning set 15 has a fourth positioning hole 151, a fifth positioning hole 152, and a sixth positioning hole 153. A second set point A2 is defined based on the second positioning slot (14) as a center. The fourth positioning hole 151, the fifth positioning hole 152 and the sixth positioning hole 153 are disposed around the second set point A2. The fourth positioning hole 151 is on the horizontal line L1, and the fifth positioning hole 152 and the sixth positioning hole 153 are disposed up and down along the vertical line L2, and located on one side of the fourth positioning hole 151. The first set point A1 and the second set point A2 are on the horizontal line L1.

The first positioning slot 12 has a first flange 121 that protrudes outwardly along a peripheral of the first positioning slot 12 and is based on the first set point A1 as a center. The first flange 121 is disposed close to the second positioning hole 132 and the third positioning hole 133. The second positioning slot 14 has a second flange 141 that protrudes outwardly along a peripheral of the second positioning slot 14 and is based on the second set point A2 as a center. The second flange 141 is disposed close to the fifth positioning hole 152 and the sixth positioning hole 153.

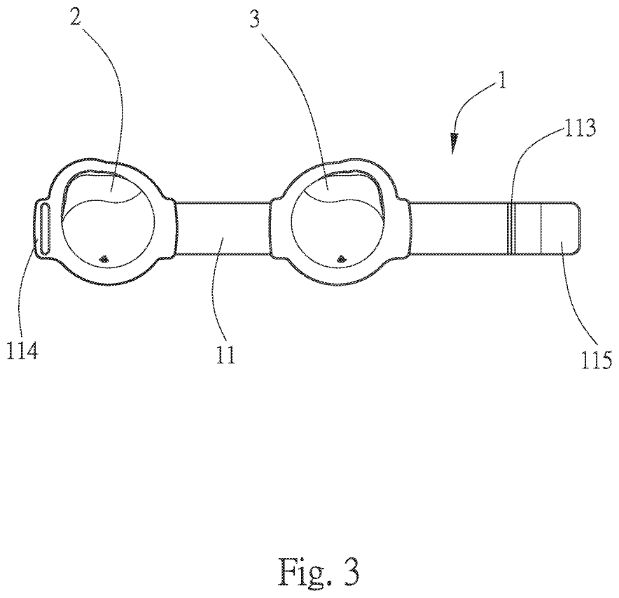

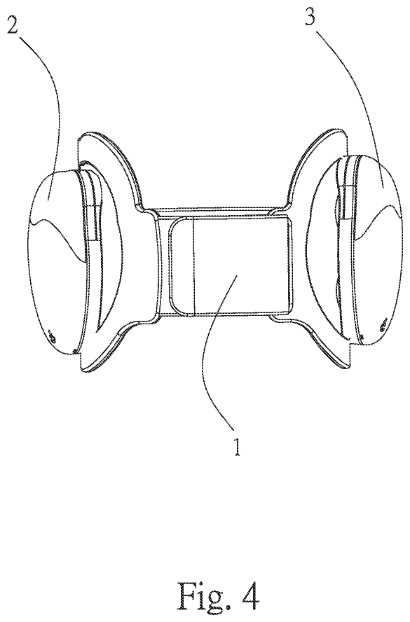

With reference to FIG. 3 and FIG. 4, the first sensing module 2 is detachably disposed on the first positioning slot 12 and has a first sensing set 21 on the first positioning set 13. The first sensing set 21 has a first sensing point 211 connecting with the first positioning hole 131, a second sensing point 212 connecting with the second positioning hole 132, and a third sensing point 213 connecting with the third positioning hole 133.

The first sensing module 2 captures biceps muscle activities. The biceps, also known as the biceps brachii, is located in front of the upper arm and consists of two arm muscles. When the elbows are flexed, the biceps then contract and bulge.

The second sensing module 3 is detachably disposed on the second positioning slot 14 and has a second sensing set 31 that is on the second positioning set 15 and held on the upper body of the wearer through one of the plural contact pad sets 6. The second sensing set 31 has a fourth sensing point 311 connecting with the fourth positioning hole 151, a fifth sensing point 312 connecting with the fifth positioning hole 152, and a sixth sensing point 313 connecting with the sixth positioning hole 153. The three contact pads of each contact pad group 6 cooperate with the fourth sensing point 311, the fifth sensing point 312, and the sixth sensing point 313 in a triangular form to hold on the upper body of the wearer.

The second sensing module 3 captures triceps muscle activities. The triceps, also known as the triceps brachii, are located at the back of the upper arm and consists of three arm muscles. When the wrist is straighten and rotated, the triceps thus contract.

By means of the first flange 121 of the first positioning slot 12 and the second flange 141 of the second positioning slot 14, they play a role to lead the wearer placing the first sensing module 2 into the first positioning slot 12, and disposing the second sensing module 3 into a fool proof setting of the second sensing module 3 as well. More particularly to be a help role that enhances the contact pad sets 6 to hold the upper body of the wearer at accurate and alignment positions.

The first sensing module 2 further has a first physiological sensing circuit, and the second sensing module 3 further has a second physiological sensing circuit. The first physiological sensing circuit, the second physiological sensing circuit and a multimedia interactive system are connected with each other in wireless, such as Bluetooth. That is, the captured all information, physiological sensing data and muscle activities, is delivered outwardly.

Additionally, the first sensing module 2 and the second sensing module 3 have a rigid case respectively for holding a whole structure and a switch for turning on and off the first sensing module 2 and the second sensing module 3. The rigid case is to accommodate other functional devices, such as physiological sensing circuit, sensor circuit, power supply unit, wireless transceiver, for structural support and protective shielding. A pocket for accommodating the first and second sensing modules 2, 3 can be provided on the first positioning groove 12 and the second positioning groove 14 respectively, therefore the pocket is a role for supporting the first and second sensing modules 2, 3 in the first positioning groove 12 and the second positioning groove 14 respectively. In practice, a Velcro/hook and loop tape is used to fix the first and second sensing modules 2, 3 individually, but not limited thereto.

For acquiring precise physiological information, the first and second sensing modules 2, 3 may be equipped different kinds of physiological sensing devices to comply the needs. For example, accelerometer, gyroscope, motion sensor, proximity sensor, optical sensor, magnetometer, pressure sensor, moisture sensor, position sensor, position tracking sensor, etc. The sensing device is selected from the group consisting of: optical sensors, magnetic sensors, inductive sensors, capacitive sensors, current sensors, resistive sensors, magnetoresistive sensors, infrared sensors, inclinometer sensors, piezoelectric material/piezoelectric based sensors, blood oxygen sensors, heart rate sensors, laser/ultrasound based sensors, and EMG type sensors. In some embodiments, physiological sensors could be configured to measure, sense position and orientation, acceleration, velocity, vibration, or shock along single or multiple axes.

In reality, the obtained muscle group data are stored in the host device, which is probably an intelligent health promotion service system (IHPSS). That is, as aforesaid, the multimedia interactive system 5 designed for muscle rehabilitation. For some embodiments, the goal of the system is to train muscles as deltoid, biceps and triceps, in order to completely rehabilitate the upper limbs to perform actions of flapping wings, bending elbows, and extending elbows. According to those trainings of muscle groups, the muscle endurance of the upper limbs/body is thus effectively improved.

The capturing user's motion data and other physiological information (for example, rehabilitation exercises) will be integrated into interactive game-type dynamic audio-visual contents, and then multimedia contents are generated. The multimedia contents are presented as a map type stage/upgrade challenge. It is to design for guiding the user through an interactive adventure virtual game, thereby reducing the fatigue of the user while stimulating self-training motivation in training.

In the embodiment, the operations of the intelligent health promotion service system can be divided into the categories of establishing basic user data, setting customized parameters, experiencing gamified rehabilitation, user feedback, and intelligently evaluating and recording, etc. On the one hand, it promotes the integration of professional rehabilitation treatment, provides remote and digital services for the elderly with frailty during the golden treatment period or health promotion period, and accurately provides the most optimal rehabilitation plan to achieve the three major service goals of “customization, gamification, and wisdom”. On the other hand, the nostalgic and retro game scene design is provided, in order to reduce the fatigue of the user, arouse the empathy of the elderly, and thus increase the motivation for rehabilitation.

Besides, during training, the condition of each muscle group of the user is inspected. The gait analysis technology is to accurately divide the gait cycle, and the degree of weakness of the rehabilitation person is then evaluated. Based on the needs of the rehabilitation person, customized game rehabilitation treatment is enhanced to generate or build up automatically, so as to construct a high-quality clinical evaluation system. Through deep learning and data analysis, the system assists professional people to create an accurate rehabilitation plan, and delivers the training conditions to the family members of the rehabilitation person. Therefore, the rehabilitation person may be treated by the plan without going outside.

Interactive somatosensory rehabilitation content is able to design training movements such as wing flapping, elbow flexion, elbow extension, etc. based on regular clinical upper limb rehabilitation training movements. Through the virtual coaching style presented by the game, rehabilitation training can help guiding the rehabilitation person to complete the rehabilitation training classes, which motivates the rehabilitation person to complete training.

For the embodiment, the AI deep learning and reinforcement learning (RL) with long and short-term memory (LSTM) are capable of evaluating the progression of the muscle endurance of the rehabilitation person. Afterward, the acquired data will be delivered to an electronic health system of the service system for analysis, and then provided to those professional people for evaluation and rehabilitation treatment reference, in order to realize the prescription of customized rehabilitation treatment courses through the real-time medical sharing system.

According to the requirements of the rehabilitation person, a rehabilitation therapy course shall be created, so as to form an interactive platform for medical sharing system. The family members of the rehabilitation person may understand the situations for him/her in a daily record, and the doctor can continuously interactive with the rehabilitation person and the family members via a professional report. That is, such rehabilitation motivation and effect may be improved.

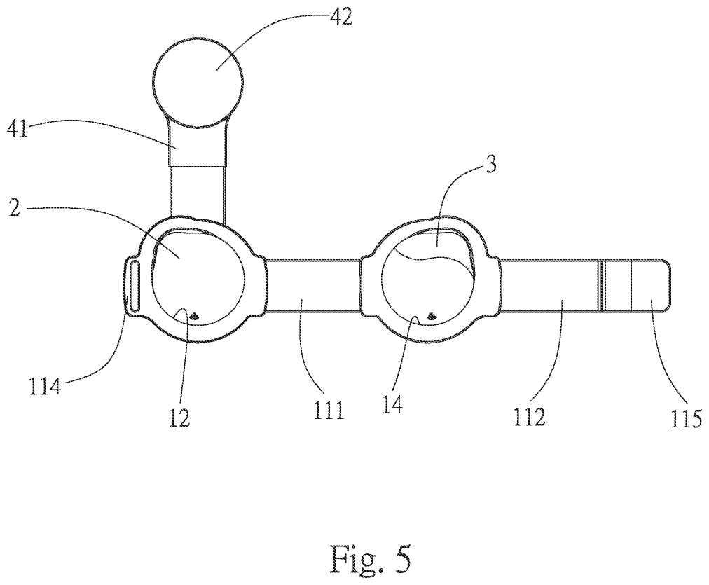

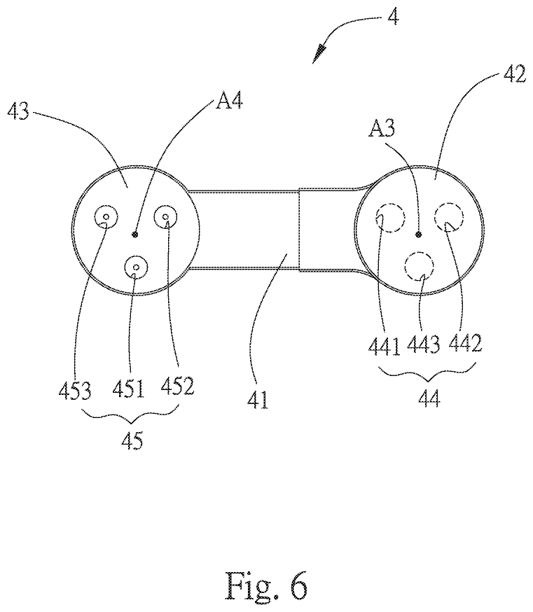

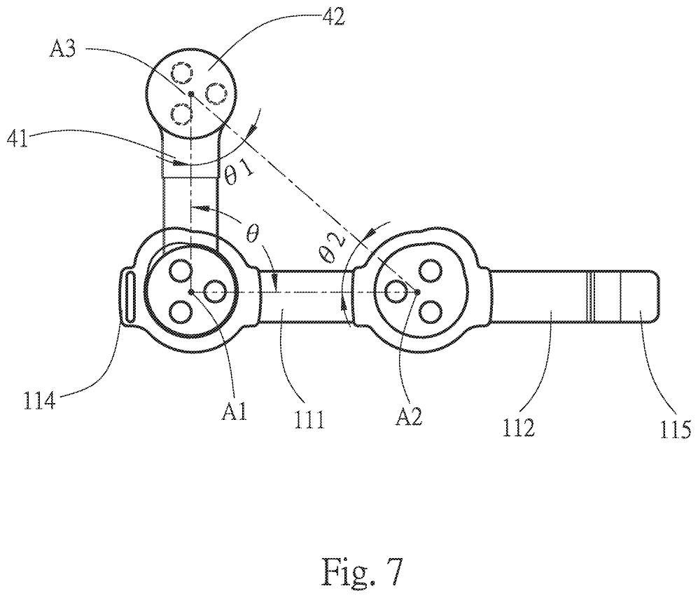

With respect to FIG. 5, FIG. 6 and FIG. 7, the extension unit 4includes an extension band 41 that detachably connects with the connection band 11, a first extension block 42 at one end of the extension band 41, a second extension block 43 at another end of the extension band 41, a first fastening set 44 on the first extension block 42, and a second fastening set 45 on the second extension block 43. When the connection band 11 connects with the extension band 41, the extension band 41 is located on the vertical line L2, then the first fastening set 44 connects with one of the plural contact pad sets 6 in order to let the first extension block 42 hold on the upper limb/body of the wearer. On the other hand, the second fastening set 45 connects with the first sensing set 21 that is on the first positioning slot 12. Via the connections of the extension band 41 and the first sensing module 2 on the connection band 11, the activities of deltoid muscle is acquired. Further, a circuit electrically connecting with the first sensing module 2 is embedded in the extension band 41 for transmitting the EMG from the activities of deltoid muscle. The position of the first extension block 42 is about 2 finger widths from the shoulder peak to the upper edge of the extension band 41. The connection band 11 and the extension band 41 are made of plastic material, and a wearing position is adjustable depending on a user's body type.

The first extension block 42 is to acquire the activities of deltoid muscle, wherein the deltoid muscle, commonly known as the “big head”, surrounds the scapula, clavicle and humerus connected by the shoulder. Most movements of the shoulder and upper arm are related to it. The deltoid muscle strengthens the shoulders and is responsible for pulling the arms forward, sideways, and backward. So, obviously it is a very important part of the body and allows the arms to move in multiple directions.

The first extension block 42, the first positioning point A1 of the first positioning slot 12, and the second positioning point A2 of the second positioning slot 14 form an isosceles triangle, wherein the distance between the first positioning point A1 and the third positioning point A3 is equal to the distance between the first positioning point A1 and the second positioning point A2, and the angles θ1 and θ2 are both 45 degrees. If the length of the connection band 11 is the maximum value, and the angle θ2 is 30 degrees, the length of the connection band 11 will make the angle θ2 be between 30˜45 degrees, and the angle θ is 90 degrees.

The connection band 11, the extension band 41 and other supporting members are made of flexible elastic fabric material or breathable material with high compressibility and non-slip properties. More, synthetic fabrics with suitable surface treatments such as antibacterial fabrics and silver fabrics are other options. Surface treatments may include water-repellent coating treatments, surface friction modification, antimicrobial surface treatments, and anti-odor surface treatments. By way of the characteristics of flexible elasticity and non-slip property, the friction and compression forces limit the movements of the connection band 11 and the extension band 41 on the upper limb/body of the wearer, so as to minimize unwanted friction, misposition, misalignment of the device, etc.

A third set point A3 is defined based on the first extension block 42 as a center, and a fourth set point A4 is defined based on the second extension block 43 as a center. The first fastening set 44 has a first fastening member 441, a second fastening member 442, and a third fastening member 443. The first fastening member 441, the second fastening member 442 and the third fastening member 443 are disposed around the third set point A3. The second fastening set 45 has a fourth fastening member 451, a fifth fastening member 452, and a sixth fastening member 453. The fourth fastening member 451, the fifth fastening member 452 and the sixth fastening member 453 are disposed around the fourth set point A4. The fourth fastening member 451, the fifth fastening member 452 and the sixth fastening member 453 can be configured as magnetic members, in order to conveniently unload the first sensing set 21.

The fourth fastening member 451 is connected with the first sensing point 211, the fifth fastening member 452 is connected with the second sensing point 212, and the sixth fastening member 453 is connected with the third sensing point 213.

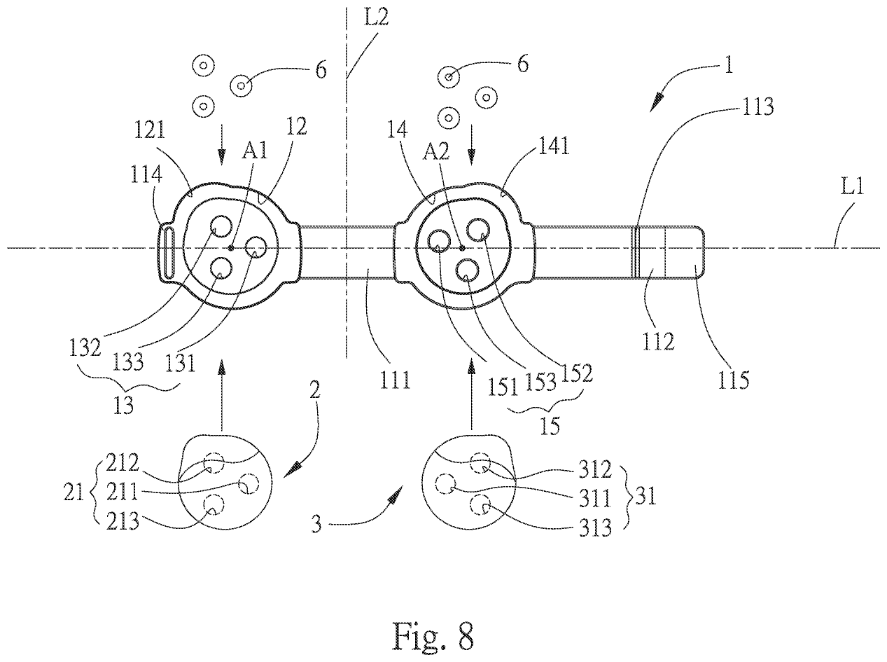

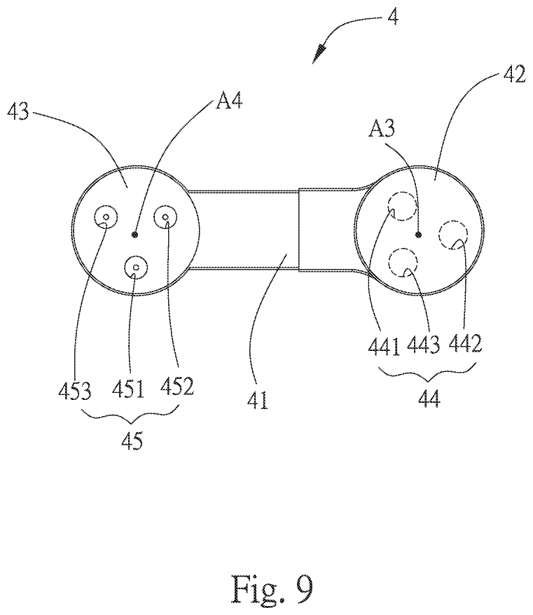

With regard to FIG. 8 and FIG. 9, which illustrate a schematic view of a second preferred embodiment of the smart wearable device of the present invention. The second preferred embodiment is the mostly same as the first preferred embodiment, and hereinafter will describe the different part. The extension band 41 is on the vertical line L2, and the first fastening member 441, the second fastening member 442 and the third fastening member 443 are rotated 30 to 50 degrees clockwise with respect to the fourth fastening member 451, the fifth fastening member 452 and the sixth fastening member 453 based on the third setting point A3 as an axis when the connection band 11 is connected with the extension band 41. The fourth positioning hole 151, the fifth positioning hole 152 and the sixth positioning hole 153 are rotated 15 to 30 degrees clockwise with respect to the first positioning hole 131, the second positioning hole 132, and the third positioning hole 133 based on the second setting point A2 as an axis. Therefore, the first positioning set 13 and the second positioning set 15 are disposed at a bilateral symmetrical interval on the horizontal line L1.

The first positioning point A1 is on the horizontal line L1, and the second positioning point A2 is disposed up and down along the vertical line L2. The angle θ in FIG. 7 is between 85 and 105 degrees, in order to generate a height difference between the first positioning set 13 and the second positioning set 15.

As a conclusion, the smart wearable device of the present invention uses the cooperations of the connection unit 1, the first sensing module 2, the second sensing module 3, and the extension unit 4 to capture the muscle activities of the deltoid via the first extension block 42 and the second sensing module 3, and the muscle activities of the biceps via the first sensing module 2, in order to detect the muscle activities of several parts in the upper limb/body. The plurality of alignment calibration lines 113 are spaced apart from each other on the upper limb/body of the wearer for universal design trends. The first flange 121 and the second flange 141 are configured to be two fool proof settings for guiding the wearer to place the first sensing module 2 and the second sensing module 3. Further, The physiological sensing data and the muscle activities captured by the first physiological sensing circuit and the second physiological sensing circuit are delivered outwardly for analysis to integrate the wearable device with the information, so as to provide sarcopenia, frailty, and elderly groups with remote and digital service systems, accurately afford the most optimal rehabilitation plan, reduce the user's experience of fatigue, and improve long-term rehabilitation training motivation for indeed reach the goal of the present invention.

Although the invention has been disclosed and illustrated with reference to particular embodiments, the principles involved are susceptible for use in numerous other embodiments that will be apparent to persons skilled in the art. This invention is, therefore, to be limited only as indicated by the scope of the appended claims

What is claimed is:

1. A smart wearable device, which is held on an upper body of a wearer by a plurality of contact pad sets, comprising:
a connection unit, having a connection band that detachably holds on the upper body of the wearer, a first positioning slot disposed on the connection band, a first positioning set disposed on the first positioning slot, a second positioning slot disposed on the connection band, and a second positioning set disposed on the second positioning slot, the connection band having a first end and a second end opposite to the first end, a horizontal line being formed from the first end to the second end, a vertical line being vertical to the horizontal line, the first positioning slot being at the first end, the second positioning slot being at the second end;
a first sensing module, detachably disposed on the first positioning slot, having a first sensing set on the first positioning set;
a second sensing module, detachably disposed on the second positioning slot, having a second sensing set that is on the second positioning set and held on the upper body of the wearer through one of the plural contact pad sets; and
an extension unit, having an extension band that detachably connects with the connection band, a first extension block at one end of the extension band, a second extension block at another end of the extension band, a first fastening set on the first extension block, and a second fastening set on the second extension block, when the connection band connects with the extension band, the extension band is located on the vertical line, then the first fastening set connects with one of the plural contact pad sets in order to let the first extension block hold on the upper body of the wearer, the second fastening set connecting with the first sensing set that is on the first positioning slot.

2. The smart wearable device according to claim 1, wherein the first positioning set has a first positioning hole, a second positioning hole, and a third positioning hole, a first set point being defined based on the first positioning slot as a center, the first positioning hole, the second positioning hole and the third positioning hole being disposed around the first set point, the first positioning hole being on the horizontal line, the second positioning hole and the third positioning hole being disposed up and down along the vertical line, and located on one side of the first positioning hole.

3. The smart wearable device according to claim 2, wherein the second positioning set has a fourth positioning hole, a fifth positioning hole, and a sixth positioning hole, a second set point being defined based on the second positioning slot as a center, the fourth positioning hole, the fifth positioning hole, and the sixth positioning hole being disposed around the second set point.

4. The smart wearable device according to claim 3, wherein the fourth positioning hole is on the horizontal line, and the fifth positioning hole and the sixth positioning hole are disposed up and down along the vertical line, and located on one side of the fourth positioning hole.

5. The smart wearable device according to claim 1, wherein the first positioning slot and the second positioning slot are disposed on the horizontal line at a bilateral symmetrical interval, and the first positioning set and the second positioning set are disposed at a bilateral symmetrical interval on the horizontal line.

6. The smart wearable device according to claim 3, wherein the first positioning slot has a first flange that protrudes outwardly along a peripheral of the first positioning slot and is based on the first set point as a center, the first flange being disposed close to the second positioning hole and the third positioning hole, the second positioning slot has a second flange that protrudes outwardly along a peripheral of the second positioning slot and is based on the second set point as a center, the second flange being disposed close to the fifth positioning hole and the sixth positioning hole.

7. The smart wearable device according to claim 1, wherein the connection band further has a plurality of alignment calibration lines at the second end, the plurality of alignment calibration lines being spaced apart from each other and arranged along the vertical line.

8. The smart wearable device according to claim 1, wherein the connection band further has a first joint member at the first end and a second joint member at the second end, the first joint member and the second joint member being connected with each other in order to hold the connection band on the upper body of the wearer.

9. The smart wearable device according to claim 1, wherein the first sensing module further has a first physiological sensing circuit, and the second sensing module further has a second physiological sensing circuit, the first physiological sensing circuit, the second physiological sensing circuit and a multimedia interactive system being connected with each other in wireless.

10. The smart wearable device according to claim 3, wherein the first sensing set has a first sensing point connecting with the first positioning hole, a second sensing point connecting with the second positioning hole, and a third sensing point connecting with the third positioning hole, and the second sensing set has a fourth sensing point connecting with the fourth positioning hole, a fifth sensing point connecting with the fifth positioning hole, and a sixth sensing point connecting with the sixth positioning hole.

11. The smart wearable device according to claim 10, wherein a third set point is defined based on the first extension block as a center, and a fourth set point is defined based on the second extension block as a center, the first fastening set having a first fastening member, a second fastening member, and a third fastening member, the first fastening member, the second fastening member and the third fastening member being disposed around the third set point, the second fastening set having a fourth fastening member, a fifth fastening member, and a sixth fastening member, the fourth fastening member, the fifth fastening member and the sixth fastening member being disposed around the fourth set point, the fourth fastening member being connected with the first sensing point, the fifth fastening member being connected with the second sensing point, and the sixth fastening member being connected with the third sensing point.

12. The smart wearable device according to claim 11, wherein the extension band is on the vertical line, and the first fastening member, the second fastening member and the third fastening member are rotated 30 to 50 degrees clockwise with respect to the fourth fastening member, the fifth fastening member, and the sixth fastening member based on the third setting point as an axis when the connection band is connected with the extension band.

13. The smart wearable device according to claim 3, wherein the fourth positioning hole, the fifth positioning hole and the sixth positioning hole are rotated 15 to 30 degrees clockwise with respect to the first positioning hole, the second positioning hole, and the third positioning hole based on the second setting point as an axis.
